(19) United States
(12) Patent Application Publication
(10) Pub. No.: US 2024/0387038 A1
(43) Pub. Date: Nov. 21, 2024

(54) AUTOMATIC PATIENT TO MEDICAL DEVICE ASSOCIATION

(71) Applicant: Hill-Rom Services, Inc., Batesville, IN (US)

(72) Inventors: Stacie Lynn Brough, Syracuse, NY (US); Anandi Mahadevan, Murrysville, PA (US)

(73) Assignee: Hill-Rom Services, Inc., Batesville, IN (US)

(21) Appl. No.: 18/668,061

(22) Filed: May 17, 2024

Related U.S. Application Data
(60) Provisional application No. 63/467,675, filed on May 19, 2023.

(51) Int. Cl. G16H 40/67; G16H 10/60

(52) U.S. Cl. CPC G16H 40/67; G16H 10/60

(57) ABSTRACT

Provided is a patient management system for sharing patient data between and among devices. A first device of a patient management system may receive, from a second device, an indication of the presence of the second device. Based at least in part on sending a request for the patient identifier to a server for information related to the second device, the first device may receive the patient identifier. The first device may then receive, from a third device, a request for the patient identifier. Based on determining that the third device is within a threshold distance from the first device, the first device may send the patient identifier to the third device.

### CROSS-REFERENCE TO RELATED APPLICATION

This application claims priority to U.S. Provisional Patent Application No. 63/467,675 (titled “Automatic Patient to Medical Device Association” and filed on May 19, 2023) which is incorporated by reference herein in its entirety.

### TECHNICAL FIELD

Provided is a patient management system, and in particular, a system configured to associate a patient with medical devices and communicate the patient association among various medical devices.

### BACKGROUND

Increasing numbers of medical devices are used to treat patients. To ensure proper care is administered, medical devices and device data needs to be associated with the correct patient. However, the process of associating patients with medical devices is inefficient and becomes increasingly burdensome as the number of medical devices and patients increases.

Current systems require expensive accessories or manual entry to associate a patient with a medical device, a process that is not only time consuming and costly, but also prone to error resulting in incorrect data being recorded for a patient, patients being misidentified, or patients being given incorrect treatment. The various example embodiments of the present disclosure are directed toward overcoming one or more of the deficiencies associated with patient management systems.

### SUMMARY

Broadly, the systems and methods disclosed and contemplated herein are directed towards a patient management system for associating one or more devices with a patient. In some examples, a first device of a patient management system may receive, from a second device of the patient management system, an indication of the presence of a second device. In some examples, the second device may be worn by the patient and may contain one or more tags. Tags may be any devices that passively or actively transmit and/or receive information from another device. For example, a tag could be a Bluetooth tag, a Bluetooth beacon, a smart device, a Wi-Fi tag, a Global Navigation Satellite System (GNSS) tag, a real-time location system (RTLS) tag, a radio frequency identification (RFID) tag, a Global Positioning System (GPS) tag, an ultrasound tag, or an infrared tag, to name a few non-limiting examples.

Once the first device receives the indication of the presence of the second device, the first device may then obtain, from the second device, patient associated information contained within the second device, for example, information in the tag. The first device may then send, to a server associated with the patient management system, a first request to receive a patient identifier associated with the patient associated tag, wherein the patient identifier contains patient information. Based at least in part on receiving the first request to receive the patient identifier, the patient management system may then send, to the first device, the patient identifier uniquely identifying the patient associated with the tag. In some examples, the first device may then receive from a third device associated with the patient management system, a second request for the patient identifier. The first device may, in some examples, determine that a distance between the first device and the third device is equal to or less than a threshold distance. Based at least in part on determining that the distance between the first device and the third device is equal to or less than the threshold distance, the first device may then send the patient identifier and/or the patient associated tag information to the third device.

Thus, this disclosure describes systems and methods which streamline processes for associating devices with patients in healthcare settings. Rather than relying on expensive and time-consuming equipment to associate a patient with each new patient device, the systems and methods described herein improve existing techniques by enabling devices to communicate patient information to and between one another, thereby preserving resources and improving patient safety within healthcare settings.

### BRIEF DESCRIPTION OF THE DRAWINGS

FIG. 1 shows a schematic block diagram of an example patient management system environment.

FIG. 2 shows a schematic block diagram of an example patient management system environment.

FIG. 3 shows a schematic block diagram for an example process for sending a patient identifier from a first device to a third device, wherein the third device includes a hospital bed.

FIG. 4 shows another schematic block diagram for an example process for sending a patient identifier from a first device to a third device, wherein the first device includes a computing device.

FIG. 5 is an example process for sending a patient identifier from a first device to a third device.

FIG. 6 is an example computing system and device which may be used to implement the described techniques.

### DETAILED DESCRIPTION

Various embodiments of the present disclosure will be described in detail with reference to the drawings, wherein like reference numerals represent like parts and assemblies throughout the several views. Additionally, any examples set forth in this specification are not intended to be limiting and merely set forth some of the many possible embodiments.

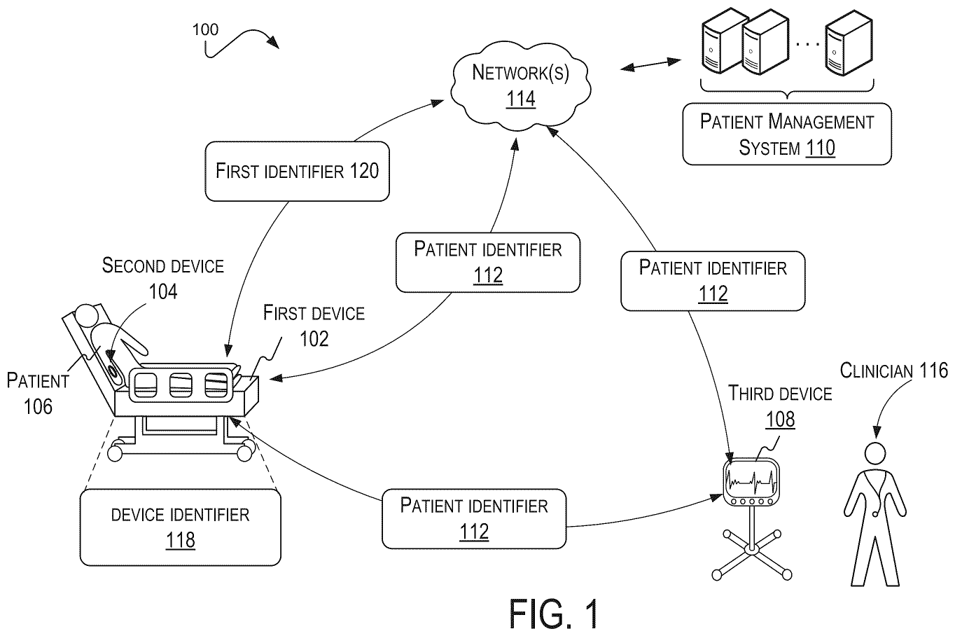

FIG. 1 is a schematic block diagram of an example patient management system environment 100 including a patient 106, a clinician 116, and at least one medical device such as first device 102, second device 104, and third device 108 in communication with a patient management system 110 via a network 114. The example patient management system environment 100 may be any healthcare environment including a clinic, doctor's office, hospital, or home healthcare setting.

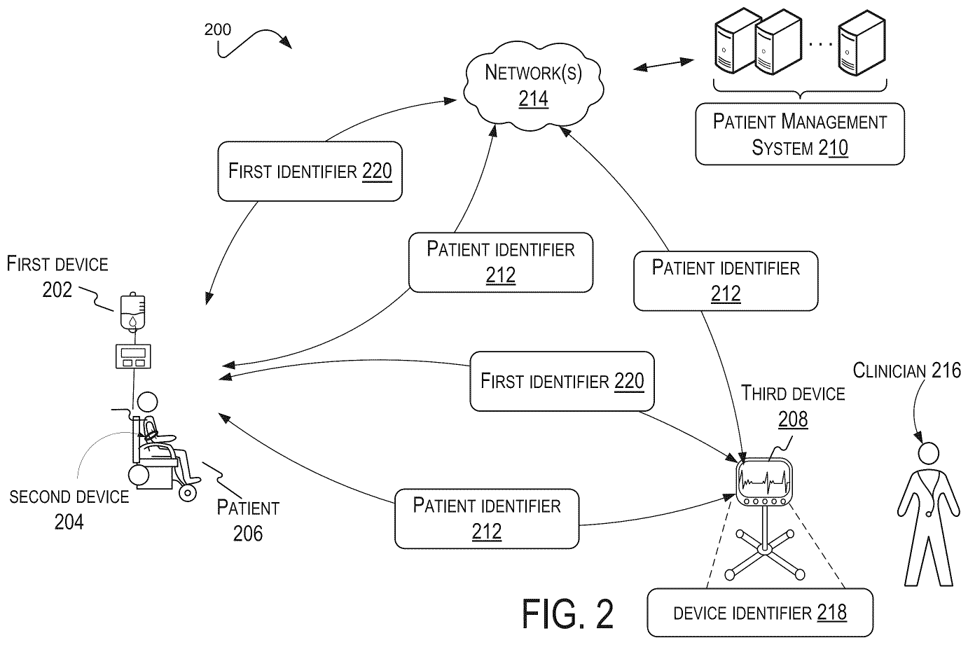

As shown in FIG. 1, the example patient management system environment 100 includes a first device 102, a second device 104, and a third device 108. While three medical devices are shown in the example patient management system environment 100, more or fewer medical devices may be present. Medical devices may be any device used in a healthcare setting including wearable devices such as pulse oximeters, blood pressure monitors, biosensors, and electrocardiogram (ECG) electrodes; a hospital bed; a pressure support surface; patient lift; lift chair; infusion pump; intracranial pressure monitor; IV pump; drains; and the like. For example, in FIG. 1, the first device 102 is a bed. In FIG. 2, the first device 202 is an infusion pump.

Generally, when a patient is admitted, they are provided with an identification device as shown by second device 104 or another component worn by the patient. In some examples, the second device 104 may be any device that includes a tag that allows for remote identification and tracking. Exemplary tags could be a Bluetooth tag, a Bluetooth beacon, a smart device, a Wi-Fi tag, a Global Navigation Satellite System (GNSS) tag, a real-time location system (RTLS) tag, a radio frequency identification (RFID) tag, a Global Positioning System (GPS) tag, an ultrasound tag, or an infrared tag, to name a few non-limiting examples.

The second device may be any type of device that may be attached to a patient including a badge, wristband, patch, clothing, or other type of accessory. Examples are also contemplated in which the second device is not necessarily “worn” on the body of the patient 106. For example, the second device 104 may be incorporated into a device such as a phone, a tablet, or a laptop computer held by the patient. The tag provides information about the patient including, for example, a patient name, date of birth, health care condition, room assignment, electronic medical records (EMR), and the like. Information on the tag may be read by a passive reader, an active reader, or a semi-active reader. In some aspects, the tags containing the first identifier 120 may be transponders which are only activated when they receive a signal from a reader, or beacons which emit signals at pre-set intervals. In some examples, the one or more transponders may include a Bluetooth beacon, a smart device, a Wi-Fi tag, a Global Navigation Satellite System (GNSS) tag, a real-time location system (RTLS) tag, an RFID tag, a Global Positioning System (GPS) tag, an ultrasound tag, or an infrared tag, to name a few non-limiting examples. The receiver may include, in some examples, a reader, a location sensor, an access point, a beacon, or a smart device, to name a few non-limiting examples. In some aspects, information may transmitted among the devices via low frequency RFID, high-frequency RFID, or ultra-high frequency RFID. In some aspects, the RFID tag uses a signal transmitted from a reader to power on.

In some aspects, a device identifier, such as device identifier 118 is incorporated into one or more other medical devices such as first device 102 and third device 108 allowing for the first device 102 and the third device 108 to detect and receive information from the second device 104. A similar device identifier 218 is shown in FIG. 2. The device identifiers, such as device identifier 118 or device identifier 218 may be an optical scanner, barcode reader, Bluetooth reader, RFID reader, QR reader, GPS reader, and the like. The device identifiers of the various medical devices may be the same or different. That is, each device may have a same or different device identifier than another device associated with the same patient, allowing the same or different communication protocols to be used. If the device identifier is a tag reader, in some aspects, the device identifier may send a signal that activates the tag. In other aspects, a beacon signal is received by reader antennas within a threshold distance of the tag. In some aspects, the tag may include a battery that is activated by a reader.

While first device 102 is depicted as a bed and third device 108 is depicted as a monitor, these devices may be any medical devices that may impact and/or monitor the health of the patient 106. Exemplary medical devices that may be used in the systems and processes described herein may include a hospital bed, a wheelchair, a walker, a blood pressure device, an SpO₂ device, a temperature device, a respiratory device, a bodyweight scale, a vision screening device, a glucometer, automated external defibrillator, a hearing screening device, a microscope, an ECG device, an overhead lift device, a pressure-sensitive mat device, a glucometer, an ultrasound machine, a drug administration system, and so on. In some aspects, the first device 102 and/or the third device 108 may include one or more computing devices such as a mobile phone, a tablet computer, a laptop computer, a desktop computer, and so forth. Alternatively or additionally, the first device 102 and/or the third device 108 may include a device used in emergency medical situations (e.g., in an ambulance and/or accessible by emergency medical technicians (EMTs).

In some examples, the medical devices may include electronic read and write capabilities such that the first device 102, the second device 104, and/or the third device 108 may be read from and/or be written on with data associated with the patient 106. For instance, the first device 102, the second device 104, and/or the third device 108 may include a processor, microprocessor, and/or other computing device components, shown and described below. For instance, the first device 102, the second device 104, and/or the third device 108 may be configured as mobile phones, tablet computers, laptop computers, etc., to deliver or communicate one or more patient identifiers amongst one another and to other devices.

In some examples, the medical devices such as first device 102, second device 104, and/or third device 108 may be enabled to communicate with one another and/or additional devices. Such communication may be radio frequency identification (RFID), near field communication (NFC), Wi-Fi, ultrawideband, Bluetooth, Zigbee, NearLink, LPWAN, cellular, Thread, and the like. For instance, the first device 102, the second device 104, and/or the third device 108 may include one or more pieces of electronic identification such as barcodes, QR codes such as two-dimensional matrix bar codes, NFC tags or chips, RTLS tags, or other passive or active identification mechanisms. While exemplary embodiments are described in terms of RFID capabilities, other types of electronic labeling and information transmission may also be used.

In some aspects, the first device 102 and/or the third device 108 may include an electronic identifier such as first identifier 120. Such an identifier may be an RFID tag, a barcode, Bluetooth tag, QR code, and the like. The devices such as first device 102 and/or third device 108 may additionally include means to read, write, receive, and transmit information electronically. In some aspects, some of the devices may include RFID capabilities and may include an RFID tag, an RFID reader, and/or an RFID antenna. It may be noted that while this application describes the use of NFC and RFID as means of communication between and among device, any wireless technology enabling the transfer of data, or combination thereof, may be used. For example, the first device 102 may receive the first identifier and/or the patient identifier from the second device 104 via an RFID and/or optical barcode scanner associated with the first device 102, but may send the first identifier and/or the patient identifier to the third device 108 via other communication modalities.

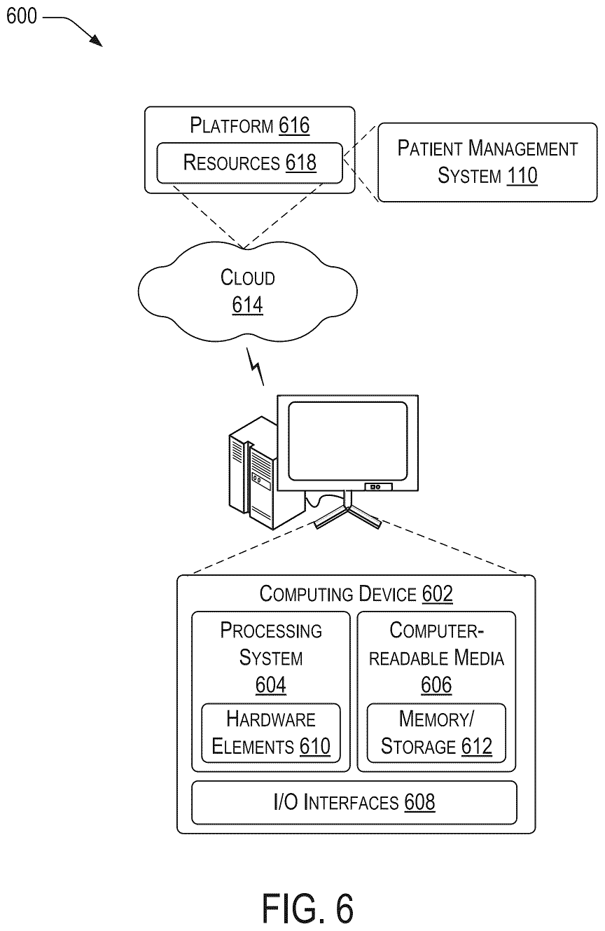

The patient management system 110 may be comprised of one or more server computing devices as described in further detail in FIG. 6 and particular systems or modules of the management system and devices may be configured to perform tasks described herein. The patient management system 110 or elements of the patient management system may communicate with the first device 102, the second device 104, and/or the third device 108 to respond to queries, receive data, and so forth. Communication between the patient management system 110, the first device 102, the second device 104, and/or the third device 108 may, in some examples, occur via the network 114, where the communication can include patient data including healthcare data such as biological data acquired by the first device 102, the second device 104, and/or the third device 108. In other aspects, other communication modalities may be used. A server of the patient management system 110 can act on the information received from the first device 102, the second device 104, and/or the third device 108, determine one or more responses, and respond back to the first device 102, the second device 104, and/or the third device 108. Alternatively, the first device 102, the second device 104 and/or the third device 108 can act on information received from one of the other devices.

The patient management system 110 may include one or more database systems accessible by a server storing different types of information. For instance, a database can store correlations and algorithms used to manage the information encompassed in the patient identifier 112 to be shared between the first device 102, the second device 104, and/or the third device 108. A database can also include clinical data. A database may reside on a server of the patient management system 110 or on separate computing device(s) accessible by the patient management system 110 either locally or in the cloud. The database may be associated with one or more healthcare establishments associated with the patient management system environment 100 and may include any database that may include patient information such as an admission, discharge, and transfer (ADT) system, an active directory, EMRs, and/or a Hospital Information System (HIS), to name a few non-limiting examples.

The network 114 or network 214 is typically any type of wireless network or other communication network known in the art. Examples of the network includes the Internet, an intranet, a wide area network (WAN), a local area network (LAN), and a virtual private network (VPN), cellular network connections and connections made using protocols such as 802.11a, b, g, n and/or ac. Alternatively or additionally, the network may include a nanoscale network, a near-field communication network, a body-area network (BAN), a personal-area network (PAN), a near-me area network (NAN), a campus-area network (CAN), and/or an inter-area network (IAN).

In some examples, the second device 104 may include a first identifier 120. The first identifier 120 may include the same or different information than the patient identifier 112. In some aspects, the first identifier 120 may include a record ID, account ID, serial number, or other unique generic identifier that has been associated with the patient and the patient's records. The patient identifier 112 may include data associated with the patient 106, such as the name, date of birth, room assignment, gender, age, medication list, health history, medical record, and the like, or any combination thereof. In some examples, the patient identifier 112 and/or first identifier 120 may be associated with a medical record of the patient 106, such as an electronic medical record (EMR).

In some aspects, information recorded by the one or more medical devices such as first device 102, second device 104, and/or third device 108 may detect patient related information such as biological data. Information detected and/or recorded by the medical devices may be transmitted via the medical devices to an EMR in, for example, the patient management system 110. In some aspects, information detected and/or recorded by the medical devices may be added to the patient identifier 112. For example, the patient identifier 112 may include the latest biological data for patient 106. Exemplary biological data may include, for example, heart rate, respiration rate, pulse, oxygenation, height, weight, electrocardiogram readings, infusion pump, urine output, temperature, blood pressure, blood glucose levels, and the like.

In some examples, the patient management system 110, the first device 102, the second device 104, and/or the third device 108 may generate, store, and/or selectively share the patient identifier 112 between one another to provide the patient 106 and/or clinicians treating the patient 106 with improved outcomes by efficiently identifying the patient 106 and providing patient information to medical devices used in treating the patient 106.

FIG. 2 shows a schematic block diagram of a patient management system environment 200 in which the first device 202 is an infusion pump associated with a patient 206 instead of a bed as shown in FIG. 1. The first device 202, the second device 204, similar to the second device 104, and the third device 208, similar to third device 108 exchange information about patient 206, similar to patient 106 between each other and also with the patient management system 210, similar to patient management system 110. In some aspects, information may be exchanged via the network 214, similar to network 114, or through other communication protocols. As shown in FIG. 2, first identifier 220, similar to first identifier 120 and patient identifier 212 similar to patient identifier 112 are acquired and exchanged between the devices and the patient management system 210, similar to patient management system 110. In some aspects, some or all of the patient information acquired or exchanged may be presented to the clinician 216, similar to clinician 116.

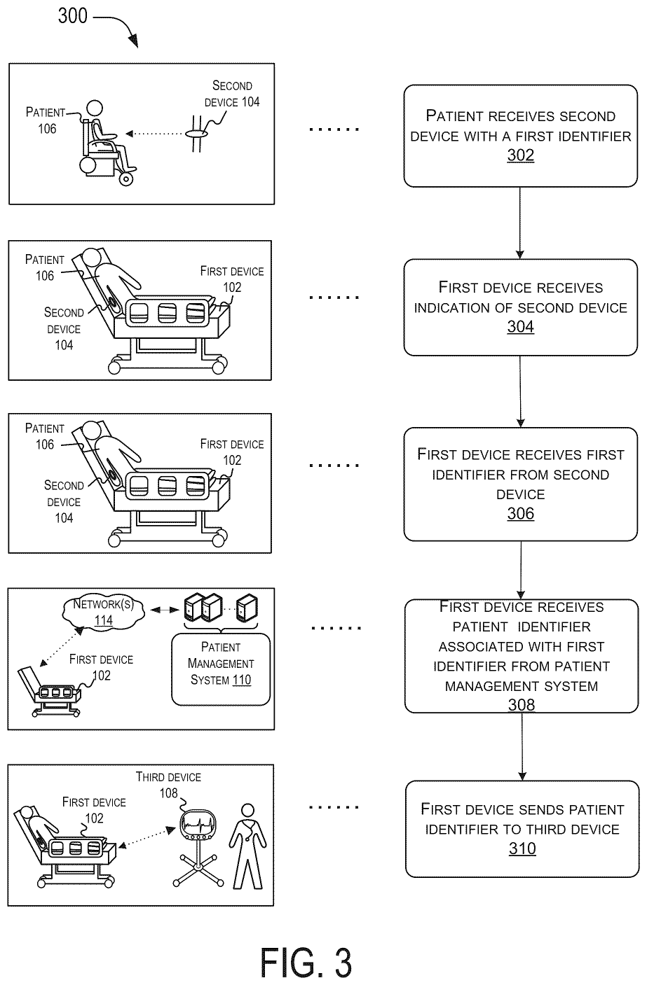

Example configurations of the first device 102, the second device 104, and/or the third device 108, as well as first device 202, second device 204, and/or third device 208 and methods for their use, are shown and described with reference to at least FIGS. 3-6 below.

FIG. 3 shows a schematic block diagram of an example process 300 for enabling a first device to receive a first identifier from a second device, receive a patient identifier, and send the patient identifier to a third device, wherein at least one of the devices includes a medical device, such as a hospital bed. The patient identifier, the first identifier, the first device, the second device and/or the third device illustrated in FIG. 3 may be the same or similar to the patient identifier 112, the first identifier 120, the first device 102, the second device 104, and/or the third device 108, respectfully, as illustrated in FIG. 1.

In some examples, as illustrated at operation 302, the process 300 may include receiving, by a patient, such as the patient 106, the second device 104 including first identifier 120. In some examples, the patient 106 may receive the second device 104 based at least in part on an admittance to a healthcare setting, such as a hospital. The second device 104 may include a first identifier 120 with patient related information such as patient identifier information 112 and/or information in the form of a generic or random number which has been associated with the patient. While the current illustration depicts the second device 104 as an armband, the second device 104 may include any device and may or may not be physically attached and/or worn by the patient 106. In some examples, the second device 104 may include RFID capabilities enabling the second device 104 to communicate with other devices having RFID capabilities. For instance, the second device 104 may include an RFID tag or chip. In other aspects, the second device 104 may include a QR code, barcode, Bluetooth beacon, or other identifying tag. In some aspects the second device 104 may include an RFID tag or chip and a QR code or barcode, allowing different medical devices to use different modalities to obtain information associated with the patient.

The second device 104 may include a first identifier including one or more ways to identify the patient. In some aspects, the first identifier is a record ID, account number, or other unique number that is associated with patient information in the patient management system 110. In other aspects, the second device 104 may include specific information about a patient such as the information contained in patient identifier 112. For example, during admittance, a healthcare provider may obtain information associated with the patient 106, such as a name of the patient 106, a date of birth of the patient 106, a gender of the patient 106, an age of the patient 106, a medication list associated with the patient 106, a location of the patient 106, a health history of the patient 106, a room assignment of a patient 106, and/or an admit status of the patient 106, to name a few non-limiting examples (collectively referred to as the “patient identifier 112”). Such information may be added to the first identifier 120 or input into the patient management system 110 and associated with first identifier 120, or both. Additionally, or alternatively, the patient 106 may already have a patient identifier 112 associated with the patient management system 110, such as an existing profile from a previous visit. In such examples, the healthcare provider may update the existing patient identifier 112 to reflect a current status of the patient 106 and add that information to the second device 104 or associate the patient identifier 112 containing the updated information with the record ID or generic number of the second device 104. For example, the second device 104 or other first identifier 120 may include, for example, Jane Doe, age 18, DOB Jan. 10, 2006. In other aspects, the second device 104 may include, for example, Jane Doe, age 18, DOB Jan. 10, 2006, admitted for shortness of breath, blood pressure 120/80 mmHg, SpO2% 75. In other aspects, the second device 104 may only contain the number 197634589 and the number 197634589 may be associated with a patient 106 and a patient identifier 112 in the patient management system 110 though different amounts and combinations of information are also contemplated.

Based at least in part on the admittance to the healthcare setting, the healthcare provider may provide, to the patient 106, the second device 104. The healthcare provider may, in some examples, input the first identifier 120 associated with the second device 104 into the patient management system 110, thus creating a digital link between the patient 106 and the second device 104 assigned to the patient.

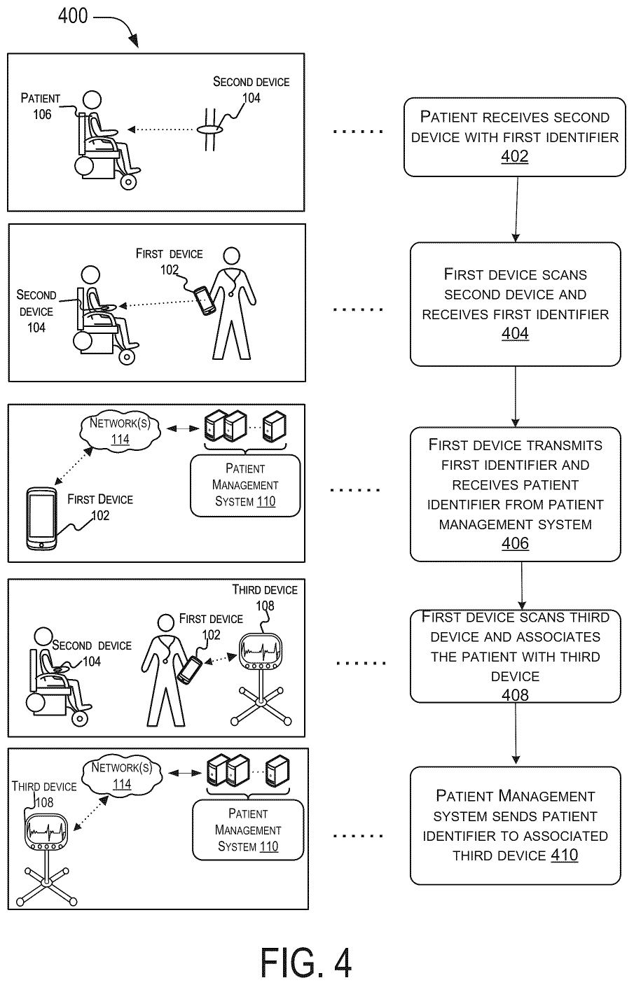

When a patient enters a patient area such as a room, the first identifier 120 may be read by a reader. Such readers, as shown for example, by device identifier 118, may be optical readers, barcode scanners, QR code scanners, Bluetooth reader, and/or an RFID reader. At operation 304, process 300 may include receiving, by the first device 102, an indication of the second device 104. That is, the first device 102 may recognize the presence of the second device 104. Such recognition may be based on passive or active behavior of the second device 104. For example, the second device 104 may only activate when in the presence of a reader. In other aspects, the second device 104 may emit a beacon signal at pre-set intervals which may be received by the first device 102. The first device 102 may, in some examples, include a medical device such as a hospital bed (as illustrated in the current embodiment), a wheelchair, a blood pressure monitor, a blood glucose monitor a pulse oximeter, or an ECG monitor, a thermometer, a respirator, to name a few non-limiting examples. In other examples, the first device 102 may include a computing device, such as mobile phone or a tablet computer. Utilizing a computing device as a first device is described in more detail in FIG. 4, below.

Similar to the second device 104, the first device 102 may include communication capabilities such that the first device 102 may be configured to exchange data with other capable devices, such as the second device 104 or third device 108. For example, the first device 102 may send a transmission which may activate the second device 104, thereby enabling the second device 104 to send information such as the first identifier and/or patient identifier to the first device 102. In some aspects, the transmission may be radio waves, though other types of signals and communication protocols may also be used. The radio waves or other signals may be receivable from a threshold distance (e.g., 1 millimeter, 5 millimeter, 10 millimeters, 1 centimeter, 10 centimeters, 1 meter, 10 meters, etc.). Based at least in part on the second device 104 being within the threshold distance of the first device, the second device 104 may receive the transmission from the first device 102, which may cause the second device 104 to send, to the first device 102, identifying information such as a first identifier 120. In some aspects, the first device 102 may receive an intermittently transmitted beacon from the second device 104. In some aspects, the first device 102 may acquire information from the second device 104 by other means, such as by scanning the second device 104 as explained in further detail in FIG. 4.

As described above, the first identifier 120 may include the patient identifier 112. However, in order to maintain the privacy of the patient 106, the second device 104 containing the first identifier 120 may, in some examples, not include the patient identifier 112. In other words, while the second device 104 may include an indication of the first identifier, the first identifier may be a standalone code. The standalone code may be sent by the second device 104 to the first device 102 at 306. Once the first device 102 receives the first identifier, the first device 102 may send, to patient management system 110 and via the network 114, a request to obtain the patient identifier 112 associated with the first identifier at operation 308. Based at least in part on inputting the first identifier 120 into the database associated with the patient management system 110, the patient management system 110 may access the patient identifier 112 and send the patient identifier 112 including an updated patient identifier 112 to the first device 102.

In some examples, the first device 102 may prompt a healthcare provider to verify the patient identifier 112 to ensure that the patient identifier 112 is indeed associated with the correct patient 106. For example, the first device 102 may include one or more user interfaces. Based at least in part on receiving the patient identifier 112, the first device 102 may cause presentation, via the one or more user interfaces, of the patient identifier. In some examples, the first device 102 may additionally or alternatively cause presentation, via the one or more user interfaces and to a healthcare provider, one or more options associated with confirm or deny the patient identifier 112.

In some examples, and as described above, multiple medical devices may be used to treat the patient 106, wherein each new device introduced may require a positive identification of the patient 106. Additional medical devices may receive the first identifier 120 and/or all or part of a patient identifier 112 from the second device 104 as described for the first device in FIG. 2 or from other devices in the patient environment that have already received the first identifier 120 or patient identifier 112. Thus, the first device 102 may send, to additional devices, the patient identifier 112, eliminating the need for a healthcare provider to manually associate each additional device to the patient 106. As such, as illustrated at operation 310, the process may include sending, from the first device 102 and to the third device 108, information related to the patient. Such information may include the first identifier and/or all or part of the information included in the patient identifier 112.

Similar to the first device 102, the third device may be a medical device, such as a wheelchair, a blood pressure device, or vital signs monitor, a blood glucose monitor, a pulse oximeter, or an ECG monitor, ventilator, to name a few non-limiting examples. The third device 108 may include, in some examples, NFC and/or RFID capabilities, enabling the third device 108 to transmit and/or transmit data to and from other NFC and RFID capable devices, such as the first device 102. In other aspects, other communication protocols may be used including Wi-Fi, ultrawideband, Bluetooth, Zigbee, NearLink, LPWAN, cellular, Thread, and the like. In some aspects, the medical devices such as first device 102, second device 104, and third device 108, may be able to communicate within threshold distances. This assists in the maintenance of patient confidentiality and prevents associating all medical devices within a care facility with a single patient, particularly in a shared room situation in which each patient may be associated with their own set of devices.

For example, the first device 102 may receive, from the third device 108, a communication such as a radio-identification or Bluetooth signal. Based at least in part on receiving the signal, the first device 102 may determine a distance between the first device 102 and the third device 108 (e.g., 1 centimeter, 10 centimeters, 1 meter, 5 meters, etc.). The first device 102 may then determine a threshold distance, wherein the threshold distance is a maximum distance between the first device 102 and the third device 108 such that the first device 102 and the third device 108 may communicate with one another. Based at least in part on determining that the distance between the first device 102 and the third device 108 is equal to or less than the threshold distance, the first device 102 may send, to the third device 108, the patient identifier 112 and/or first identifier 120. In the event that the distance between the first device and the second device is greater than a threshold distance, the patient identifier 112 and/or the first identifier 120 will not be sent. While such transmissions are described as using radio waves as part of an RFID system, other communication protocols may also be used to send and receive signals.

While the current embodiment illustrates the first device 102 and the third device 108, any number of devices may be used. Moreover, in some examples, any device associated with the patient 106 may send, to an additional device, the first identifier associated with the patient and/or all or part of the patient identifier 112. For example, the first device 102 and/or the third device 108 may receive, from a fourth device, a request for the patient identifier 112. The fourth device may be the same or similar to the first device 102 and the third device 108 and may include NFC and/or RFID capabilities or other communication protocols. Similar to that described above with respect to operation 308, the first device 102 and/or the third device 108 may receive, from the fourth device, a signal such as a radio-identification signal, though other types of signals may also be received. Based at least in part on receiving the signal and determining that a distance between the first device 102 and/or the third device 108 and the fourth device is equal to or less than a threshold distance, the first device 102 and/or the third device 108 may send, to the fourth device and via one or more signals associated with the first device 102 and/or the third device, the patient identifier 112. In this way, multiple devices associated with the patient 106 may communicate between and among one another, enabling a seamless transfer of patient information.

In some examples, the patient identifier 112 may be modified while the first device 102 and/or the third device 108 are still associated with the patient 106 (i.e., in use). Thus, in order to ensure that healthcare providers are being provided with the most current patient information, the patient management system 110 may send, to the first device 102 and/or the third device 108, an updated patient identifier 112. This may include things such as, but not limited to, an update to the patient care information, a healthcare provider shift change, an assignment of a new healthcare provider to the patient 106, device readings of a patient 106, an administration of a medicine to the patient 106, an update to a medication list of the patient 106, a transfer of the patient 106, a change in an admit status of the patient, an addition of a new device, and/or a removal of an existing device, to name a few non-limiting examples. In some examples, the modified patient identifier 112 may be automatically sent from the patient management system 110 to the first device 102 and/or the third device 108. For example, the patient management system 110 may receive an occurrence of an event such as a new medical device reading. The event may result in an update to the patient identifier 112 with the additional information. The update may result in the modified patient identifier 112 being sent from the patient management system 110 to the first device 102 and/or the third device 108. In some aspects, the server may provide an indication or a notification that the patient identifier 112 has been updated and the first device 102 and/or third device 108 may request the updated patient identifier 112 as needed. In some aspects, the update may be sent to either the first or third medical device and the medical device that has received the update may send the update to other medical devices associated with the patient.

Additionally or alternatively, once the first device 102 and/or the third device 108 have received the first identifier, they may send, to the patient management system 110, a request for the patient identifier 112. In some examples, the request for the patient identifier 112 may be sent periodically at predetermined intervals of time (e.g., every minute, every 5 minutes, every 10 minutes, every hour, every 12 hours, every day, etc.). Similar to that described above at operation 306, the first device 102 and/or the third device 108 may send, to the patient management system 110, a request for the patient identifier 112. Based at least in part on receiving the request, the patient management system 110 may send, to the first device and/or the third device, the patient identifier 112. In some aspects, the patient identifier 112 may be sent from the patient management system 110 only when new information is added to the patient identifier 112. Thus, if a request is received from one of the first device 102 and/or the third device 108 after they have initially received the patient identifier 112, the information will not be re-written unless new information has been added to the patient management system 110.

In some examples, the patient 106 may no longer require the first device 102 and/or the third device 108, enabling the first device 102 and/or the third device 108 to be used to care for another patient. However, because the first device 102 and/or the third device may be associated with the patient identifier 112, it may be desirable to disassociate the first device 102 and/or the third device 108 from the first identifier 120 in the second device 104. Thus, the second device 104 may, in some examples, disassociate from the first device 102 and/or the third device 108 by removing the first identifier and/or the patient identifier from the memory of the first device 102 and/or the third device 108.

In some examples, the second device 104 may disassociate from the first device 102 and/or the third device 108 based on receiving an input from the patient management system 110. For example, the patient management system 110 may receive an indication that the first device 102 and/or the third device 108 no longer needs to be associated with the second device. Based at least in part on receiving the indication of the signal, the patient management system 110 may then send, to the first device 102 and/or the third device 108, an indication to disassociate the patient identifier 112 and/or first identifier 120. Disassociating the patient identifier may include, for example, deleting the patient identifier 112 from the memory of the first device 102 and/or the third device 108. In other examples, disassociation may be performed manually at the first device 102 and/or the third device 108, such as by a clinician, or automatically via an admission-discharge-transfer (ADT) system. In some aspects, instructions to disassociate may be sent from one medical device to another medical device associated with the patient. Additionally, in some examples, patient data associated with the patient identifier may be deleted from the memory of the second device 104 automatically and/or manually upon discharge.

FIG. 4 shows another schematic block diagram of an example process 300 for enabling a first device to receive a patient identifier from a second device and transmit the patient identifier to a third device, wherein the first device includes a smart device. The first identifier, the patient identifier, the first device, the second device, and/or the third device illustrated in FIG. 4 may be the same or similar to the first identifier 120, the patient identifier 112, the first device 102, the second device 104, and/or the third device 108, respectfully, illustrated in FIG. 1.

In some examples, as illustrated at operation 402, the process 400 may include at least receiving, by a patient, such as the patient 106, the second device 104, where the second device 104 includes a first identifier. A first identifier may be information recorded on a tag such as a patient identifier 112 or a record locator or first identifier 120 which has been associated with the patient. Operation 402 may be the same or similar to operation 302, described above in FIG. 3.

At operation 404, the process 400 may include at least scanning, by the first device 102, the second device 104. For example, and as illustrated in the current embodiment, the first device 102 may, in some examples, include a computing device, such as a mobile phone, a tablet computer, a laptop computer, or a desktop computer, to name a few non-limiting examples. In some examples, the first device 102 may include one or more scanning components capable of receiving a code, such as a barcode, a quick response (QR) code, or an RFID code. In some examples, scanning the second device 104 may include a scannable identifier such that, based at least in part on scanning the scannable identifier, the first device may receive the first identifier 120 associated with the second device 104.

At operation 406, the process 400 may include at least sending, from the first device 102 and to the patient management system 110, a request to obtain the patient identifier 112 associated with the first identifier 120. In some aspects, the first device 102 may send patient identifier 112 to the patient management system 110 and receive an updated patient identifier 112. Alternatively or concurrently, the first device 102 may transmit a first identifier for the second device 104 such as a record locator or other generic identifier that has been associated with the patient 106 in the patient management system 110, but does not include specific details regarding the patient.

As described above, the third device 108 may request, from the first device 102, patient information. For example, the third device 108 may include codes similar to the scannable code or tag of the second device 104. The first device 102 may scan the third device and associate the patient with the third device at operation 408. In some examples, the first device 102 may cause presentation, via one or more user interfaces of the first device 102, of one or more options associated with approving the request to send the patient identifier 112 to the third device 108. Based at least in part on receiving an indication of an approval of the request to send the first identifier 120 to the third device 108, the first device 102 may send the first identifier 120 to the third device 108. In other examples, the first device 102 may automatically send the first identifier 120 to the third device 108 based at least in part on receiving the request, from the third device 108, for the first identifier 120. In some aspects, the first device 102 may send a patient identifier 112. Once the third device 108 has been associated with a particular patient 106, the patient management system 110 or the first device 102 may send patient related information to the third device at operation 310. Such information may include the first identifier, and/or all or part of patient identifier 112.

In some examples, the third device 108 may include one or more scanning devices, similar to the first device 102, such that the third device 108 may scan codes associated with additional devices to send, to the additional devices, first identifier 120 and/or the patient identifier 112, similar to that described above with respect to FIG. 3.

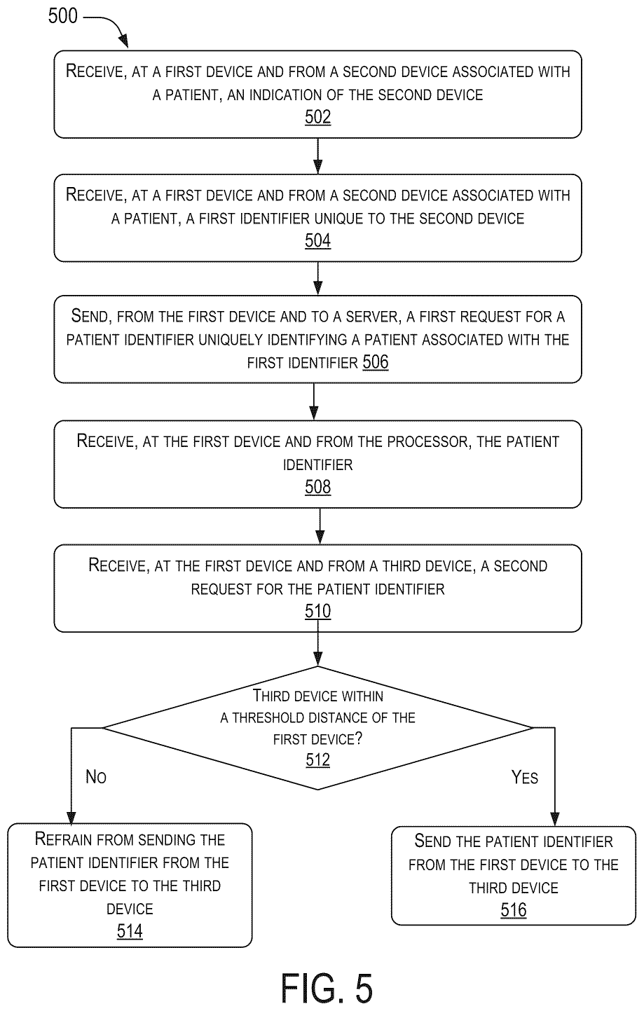

FIG. 5 is an example process 400 for sending a patient identifier from a first device to a third device, according to the techniques described herein. In some examples, the process 500 may be performed by one or more processors of computing devices, such as the patient management system 110 of FIG. 1

At operation 502, the process 500 may include receiving, at a first device and from a second device associated with a patient, an indication of the second device. That is, the first device 102 may recognize the presence of the second device 104. Such recognition may be based on passive or active behavior of the second device 104. For example, the second device 104 may only activate when in the presence of a reader. In other aspects, the second device 104 may emit a beacon signal at pre-set intervals which may be received by the first device 102. The first device may include any medical device which may generally exist in a healthcare establishment (e.g., doctor's office, hospital, clinic, dentist's office, pharmacy, ambulance, and the like) that may impact and/or monitor the health of the patient. However, the first device is not limited to devices solely existing in a healthcare establishment. For example, the first device may include one or more computing devices, such as a tablet computer, a desktop computer, and so forth.

In some examples, the first device may include radio-frequency capabilities, such as NFC, enabling short-range communication. For example, the first device may include one or more NFC tags or chips. Additionally or alternatively, the first device may include RFID capabilities, and may include an RFID tag, and RFID reader, and/or an RFID antenna though other communication protocols may also be used such as Bluetooth, Wi-Fi, GPS, RTLS, and the like to name a few non-limiting examples.

In some examples, the second device may be the same or similar to the first device, and may include NFC and/or RFID capabilities. In other aspects, the second device may include or present information according to a different or additional protocol. For example, the second device may include an RFID tag, which may be associated with an RFID identifier unique to the second device. In other aspects, the second device may include a barcode, QR code, or other readable information. In some aspects, the second device may include both an RFID and other information source, allowing different other medical devices to interact with the second medical device using different communication protocols.

The second device may be, in some examples, wearable by the patient, such as an armband or a wristband. In some examples, the first device may transmit one or more radio waves or other signals, which may activate the second device, causing the second device to send the first identifier to the first device. In other aspects, the second device may be scanned or otherwise transmit information.

At operation 504, the process 500 may include receiving, from the second device associated with the patient, a first identifier unique to the second device. Such a first identifier may be a patient identifier 112, or a record or other generic number that has been previously associated with the patient identifier 112 in the patient management system 110.

At operation 506, the first device such as first device 102 sends a request to a server, for a patient identifier 112 or updated patient identifier 112 uniquely identifying a patient associated with the first identifier. In some examples, the server may include a patient management system, which may be accessible by the first device and/or the second device. The patient management system may include one or more databases associated with one or more healthcare establishments, such as an ADT system or an EMR system. The one or more databases may include patient information, such as a name of the patient, a date of birth of the patient, a gender of the patient, an age of the patient, a medication list of the patient, a location of the patient, a room assignment, a health history of the patient, and/or an admit status of the patient, to name a few examples (collectively referred to as a “patient identifier.”)

For example, based at least in part on receiving the indication of the second device, including the first identifier associated with the second device, the first device may send, to the server, a request for the patient identifier associated with the first identifier associated with the second device. Thus, at operation 508, the process 500 may include receiving, at the first device and from the processor, the patient identifier.

At operation 510, the process 500 may include receiving, at the first device and from the third device, a second request for the patient identifier. The third device may be the same or similar to the first device in that the third device may be, but is not limited to, any medical device which may generally exist within a healthcare establishment. Similarly, the third device may include radio-frequency capabilities, such as NFC and RFID or other transmission capabilities such as, for example, Bluetooth or Wi-Fi. For example, the first device may receive one or more radio-identification signals or Bluetooth signals from the third device.

Based at least in part on receiving the one or more radio-identification signals, the first device may determine a distance between the first device and the third device. At operation 410, the process 400 may include determining, by the first device, that the third device is within a threshold distance of the first device. For example, the first device may determine the distance between the first device and the third device (e.g., 1 centimeter, 10 centimeters, 1 meter, 4 meters, etc.). The first device may then determine a threshold distance, wherein the threshold distance is a maximum distance between the first device and the third device such that the first device and the third device may communicate with one another. In some examples, the threshold distance may be determined based at least in part on a strength of a wireless signal associated with the first device and/or the third device. For example, the first device may determine a strength of a signal associated with the third device, wherein a stronger or a larger signal may be associated with a smaller threshold distance, whereas a weaker or a smaller signal may be associated with a larger threshold distance.

Additionally, or alternatively, the threshold distance may be determined utilizing a real-time locating systems (RTLS). For example, the first device, the second device, and/or the third device may include one or more transponders which may be configured to be identified by one or more receivers. In some examples, the one or more transponders may include a Bluetooth beacon, a smart device, a Wi-Fi tag, a Global Navigation Satellite System (GNSS) tag, a Global Positioning System (GPS) tag, an ultrasound tag, or an infrared tag, to name a few non-limiting examples. The receiver may include, in some examples, a reader, a location sensor, an access point, a beacon, or a smart device, to name a few non-limiting examples. In some examples, the one or more receivers may determine the location of the one or more transponders using tracking systems such as wi-fi signals, GNSS, GPS, ultrasound, or Bluetooth technology, to name a few non-limiting examples. Based at least in part on determining the location of the transponder associated with the device, a distance may be determined between the first device and the third device, wherein the threshold distance may be based at least in part on the distance between the first device and the third device.

Additionally, it may be noted that while the current operation describes the first device as determining the threshold distance, this may be additionally or alternatively determined by other devices, such as the third device.

Based at least in part on determining that the third device is not within the threshold distance of the first device (e.g., “No” at operation 512), the process 500 may include, at operation 514, refraining from sending the patient identifier from the first device to the third device. In some examples, the first device may, after a predetermined period of time, re-determine the distance between the first device and the third device, repeating operation 512.

Alternatively, the first device may determine that the third device is within the threshold distance of the first device (e.g., “Yes” at operation 512). Thus, the process 500 may include, at operation 516, sending the patient identifier from the first device to the third device. For example, based at least in part on determining that the distance between the first device and the third device is equal to or less than the threshold distance, the first device may send, via radio-waves, the patient identifier to the third device.

FIG. 6 is an example computing system and device which may be used to implement the described techniques and perform one or more of the operations described herein.

### Example System and Device

FIG. 6 illustrates an example system generally at 600 that includes an example computing device 602 that is representative of one or more computing systems and/or devices that may implement the various techniques, modules, and flowcharts described herein. This is illustrated through inclusion of the patient management system 110. The computing device 602 may be, for example, a server of a service provider, a device associated with a client (e.g., a client device), an on-chip system, and/or any other suitable computing device or computing system.

The example computing device 602 as illustrated includes a processing system 604, one or more computer-readable media 606, and one or more I/O interface 608 that are communicatively coupled, one to another. Although not shown, the computing device 602 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 604 is representative of functionality used to perform one or more operations described herein using hardware. Accordingly, the processing system 604 is illustrated as including hardware element 610 that may be configured as processors, functional blocks, and so forth. This may include implementation in hardware as an application specific integrated circuit or other logic device formed using one or more semiconductors. The hardware elements 610 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable storage media 606 is illustrated as including memory/storage 612. The memory/storage 612 represents memory/storage capacity associated with one or more computer-readable media. The memory/storage component 612 may include volatile media (such as random access memory (RAM)) and/or nonvolatile media (such as read only memory (ROM)), Flash memory, optical disks, magnetic disks, and so forth). The memory/storage component 612 may include fixed media (e.g., RAM, ROM, a fixed hard drive, and so on) as well as removable media (e.g., flash memory, a removable hard drive, an optical disc, and so forth). The computer-readable media 606 may be configured in a variety of other ways as further described below.

Input/output interface(s) 608 are representative of functionality to allow a user to enter commands and information to computing device 602, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 602 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” “logic,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules, flowcharts, and techniques may be stored on and/or transmitted across some form of computer-readable media. The computer-readable media may include a variety of media that may be accessed by the computing device 602. By way of example, and not limitation, computer-readable media may include “computer-readable storage media” and “computer-readable transmission media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer-readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable transmission media” may refer to a medium that is configured to transmit instructions to the hardware of the computing device 602, such as via a network. Computer-readable transmission media typically may transmit computer-readable instructions, data structures, program modules, or other data in a modulated data signal, such as carrier waves, data signals, or other transport mechanism. Computer-readable transmission media also include any information delivery media. The term “modulated data signal” means a signal that has one or more of its characteristics set or changed in such a manner as to encode information in the signal. By way of example, and not limitation, computer-readable transmission media include wired media such as a wired network or direct-wired connection, and wireless media such as acoustic, radio frequency (RF), infrared, and other wireless media.

As previously described, hardware elements 610 and computer-readable media 606 are representative of modules, programmable device logic and/or device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 610. The computing device 602 may be configured to implement particular instructions and/or functions corresponding to the software and/or hardware modules. Accordingly, implementation of a module that is executable by the computing device 602 as software may be achieved at least partially in hardware, e.g., through use of computer-readable storage media and/or hardware elements 610 of the processing system 604. The instructions and/or functions may be executable/operable by one or more articles of manufacture (for example, one or more computing devices 602 and/or processing systems 604) to implement techniques, modules, and examples described herein.

The techniques described herein may be supported by various configurations of the computing device 602 and are not limited to the specific examples of the techniques described herein. This functionality may also be implemented all or in part through use of a distributed system, such as over a “cloud” 614 via a platform 616 as described below.

The cloud 614 includes and/or is representative of a platform 616 for resources 618. The platform 616 abstracts underlying functionality of hardware (e.g., servers) and software resources of the cloud 614. The resources 618 may include applications and/or data that can be utilized while computer processing is executed on servers that are remote from the computing device 602. Resources 618 can also include services provided over the Internet and/or through a subscriber network, such as a cellular or Wi-Fi network.

The platform 616 may abstract resources and functions to connect the computing device 602 with other computing devices. The platform 616 may also be scalable to provide a corresponding level of scale to encountered demand for the resources 618 that are implemented via the platform 616. Accordingly, in an interconnected device embodiment, implementation of functionality described herein may be distributed throughout multiple devices of the system 600. For example, the functionality may be implemented in part on the computing device 602 as well as via the platform 616 which may represent a cloud computing environment 614.

The example systems and methods of the present disclosure overcome various deficiencies of known prior art devices. Other embodiments of the present disclosure will be apparent to those skilled in the art from consideration of the specification and practice of the disclosure contained herein. It is intended that the specification and examples be considered as example only, with a true scope and spirit of the present disclosure being indicated by the following claims.

### Example Clauses

1. A method, including: receiving, at a first device and from a second device, an indication of a presence of the second device; receiving, at the first device and from the second device, a first identifier unique to the second device; sending, from the first device and to a server, a first request, including the first identifier, for a patient identifier uniquely identifying a patient associated with the first identifier; receiving, at the first device and from the server, the patient identifier; receiving, at the first device and from a third device, a second request for the patient identifier; determining that the third device is within a threshold distance from the first device; and based on the second request, and on determining that the third device is within the threshold distance, sending the patient identifier from the first device to the third device.

2. The method of clause 1, wherein sending the patient identifier to the third device further includes: scanning, with a scanning component of the first device, an identifier associated with the third device; receiving, at the first device and based on scanning the identifier associated with the third device, a request for the patient identifier from the third device; and sending, from the first device and based on receiving the request for the patient identifier, the patient identifier to the third device.

3. The method of clause 1 or 2, further including: receiving, from a fourth device, a third request for the patient identifier; based on the third request, determining that the third device is within the threshold distance from the first device; based on the third request, determining that the fourth device is within the threshold distance from the first device; and based on determining that the third device and fourth device are within the threshold distance, sending, from the first device or the third device, the patient identifier to the fourth device.

4. The method of any of clauses 1 to 3 further including: receiving, at the third device, biological data associated with the patient; sending, from the third device to the server, the biological data; and receiving, at the third device, based on sending the biological data, and from the server, an updated patient identifier including the biological data.

5. The method of any of clauses 1 to 4, wherein determining that the third device is within the threshold distance of the first device includes: receiving, at the first device and from the third device, a first signal; determining, by the first device and based on the first signal, a distance between the first device and the third device; and determining, by the first device, that the distance between the first device and the third device is less than or equal to the threshold distance; wherein the patient identifier is sent to the third device is based at least in part on determining that the distance between the first device and the third device is less than or equal to the threshold distance.

6. The method of any of clauses 1 to 5, further including: receiving, at the first device and from the server, a notification of an update to the patient identifier; sending, from the first device and to the server, a request for the updated patient identifier; receiving, at the first device, the updated patient identifier; and causing presentation, via a user interface of the first device, of the updated patient identifier.

7. The method of any of clauses 1 to 6, wherein the second device is an RFID tag, a barcode, a two-dimensional matrix barcode, a real-time location system tag, or a global positioning tag.

8. The method of any of clauses 1 to 7, wherein the first device includes a tag reader, and wherein receiving the first identifier from the second device includes the first device receiving a signal from the second device.

9. The method of any of any of clauses 1 to 8, wherein the patient identifier includes at least one of: a name of the patient; a date of birth of the patient; a gender of the patient; an age of the patient; a medication list of the patient; a room number of a patient; a location of the patient; a health history of the patient; an admit status of the patient; a medical record number; or a visit number.

10. The method of any of clauses 1 to 9, further including: sending, from the first device and to the server, a third request for the patient identifier; receiving, from the server, by the first device, and based on the third request, a disassociation indication; and removing, based on the disassociation indication and from a memory of the first device, the first identifier and the patient identifier.

11. A system, including: one or more processors; and one or more computer-readable media storing instructions that, when executed by the one or more processors, cause the one or more processors to perform operations including: receiving, at a first device and from a second device, an indication of a presence of the second device; receiving, at the first device and from the second device, a first identifier unique to the second device; sending, from the first device and to a server, a first request including the first identifier for a patient identifier uniquely identifying a patient and associated with the first identifier; receiving, at the first device and from the server, the patient identifier; receiving, at the first device and from a third device, a second request for the patient identifier; determining, based on the second request, that the third device is within a threshold distance from the first device; and based on determining that the third device is within the threshold distance, sending the patient identifier from the first device to the third device.

12. The system of clause 11, wherein sending the patient identifier to the third device further includes: scanning, with a scanning component of the first device, an identifier associated with the third device; receiving, at the first device and based on scanning the identifier associated with the third device, a request for the patient identifier; and sending, from the first device and based on receiving the request for the patient identifier, patient information to the third device.

13. The system of clause 11 or 12, the operations further including: receiving, from a fourth device, a third request for the patient identifier; based on the third request, determining that the third device is within the threshold distance from the first device; based on the third request, determining that the fourth device is within the threshold distance from the third device; and based on determining that the third device is within the threshold distance, sending, from the first device or the third device, the patient identifier to the fourth device.

14. The system of any of clauses 11 to 13, wherein determining that the third device is within the threshold distance of the first device includes: receiving, at the first device and from the third device, a first signal; determining, by the first device and based on the first signal, a distance between the first device and the third device; and determining, by the first device, that the distance between the first device and the third device is less than or equal to the threshold distance; wherein the patient identifier is sent to the third device is based at least in part on determining that the distance between the first device and the third device is equal to or less than the threshold distance.

15. A method, including: scanning, with a first device, a tag of a second device including; receiving, at the first device and from the second device, a first identifier unique to the tag of the second device; sending, from the first device and to a server, a first request for a patient identifier uniquely identifying a patient associated with the first identifier; receiving, at the first device and from the server, the patient identifier; scanning, with a scanning component of the first device, a second identifier associated with a third device; receiving, at the first device and based on scanning the second identifier associated with the third device, a second request for the patient identifier from the third device; and sending, from the first device and based on receiving the second request for the patient identifier, the patient identifier to the third device.

16. The method of clause 15, wherein the tag is a radio frequency identification (RFID) tag, a barcode, a two-dimensional matrix barcode, Bluetooth tag, a real time location system (RTLS) tag, or a geolocation tag.

17. The method of clause 15 or 16, wherein the tag includes a unique identifier associated with a patient in the server.

18. The method of any of clauses 15 to 17, wherein the patient identifier includes at least one of: a name of the patient; a date of birth of the patient; a gender of the patient; an age of the patient; a room number of a patient; a medication list of the patient; a location of the patient; a health history of the patient; an admit status of the patient; a medical record number; or a visit number.

19. The method of any of clauses 15 to 18, further including: recording, via the first device, biological data of the patient; sending, from the first device to the server, the biological data; and receiving, at the first device based on sending the biological data and from the server, an updated patient identifier including the biological data.

20. The method of any of clauses 15 to 19, further including: receiving, at the first device, a third request for the patient identifier from a fourth device; determining, based on the third request from the fourth device, that the fourth device is within a threshold distance from the first device; and based on determining that the fourth device is within the threshold distance from the first device, sending the patient identifier from the first device to the fourth device.

21. A method of associating and disassociating a patient identifier with multiple devices using a server, the method including determining that at least two devices of the multiple devices are within a threshold distance of one another; and sending the patient identifier to at least one of the multiple devices via the server.

22. The method of clause 21, wherein at least one of the at least two devices is: a smart device; a wheelchair; a hospital bed; a vital signs monitor; an infusion pump; a blood pressure monitor; a blood glucose monitor; a pulse oximeter; or an electrocardiogram (ECG) monitor.

23. The method of clause 21 or 22, wherein determining that the at least two devices of the multiple devices are within the threshold distance is based at least in part on a radio-identification signal generated by one of the at least two devices.

24. A method of associating a patient identifier of a patient with multiple devices using a server, the method including associating a first device with the patient identifier via the server and sending the patient identifier from the first device to a second device based on determining that the second device is within a threshold distance of the first device.

25. The method of clause 24, wherein determining that the second device is within the threshold distance of the first device is based at least in part on determining, by the first device, that a distance based at least in part on a radio-identification signal from the second device is less than or equal to the threshold distance.

26. The method of clause 24 or 25, wherein the patient identifier includes at least one of: a name of the patient; a date of birth of the patient; a gender of the patient; an age of the patient; a room number of a patient; a medication list of the patient; a location of the patient; a health history of the patient; an admit status of the patient; a medical record number; or a visit number.

27. A method of including: receiving, by a first device, from a component worn by a patient, and based on the component being disposed less than a first threshold distance from the first device, information unique to the component; providing, by the first device, the information to a server separate from the first device.

28. The method of clause 27 further including: receiving, by the first device, from the server, and based on providing the information, a patient identifier unique to the patient; and providing, by the first device, the patient identifier to a second device based on the second device being disposed less than a second threshold distance from the first device.

29. The method of clause 27 or 28, wherein at least one of the first device and the second device includes at least one of: a smart device; a wheelchair; a hospital bed; an infusion pump; a vital signs monitor; a blood pressure monitor; a blood glucose monitor; a pulse oximeter; or an electrocardiogram (ECG) monitor.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components unless the context requires otherwise.

As used herein, the term “based on” can be used synonymously with “based, at least in part, on” and “based at least partly on.”

As used herein, the terms “comprises/comprising/comprised” and “includes/including/included,” and their equivalents can be used interchangeably. An apparatus, system, or method that “comprises A, B, and C” includes A, B, and C, but also can include other components (e.g., D) as well. That is, the apparatus, system, or method is not limited to components A, B, and C.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and website content throughout this specification (referenced materials herein). Each of the referenced materials is individually incorporated herein by reference in their entirety for their referenced teaching(s), as of the filing date of this application.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when the application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

Although the subject matter has been described in language specific to structural features and/or methodological acts, it is to be understood that the subject matter defined in the appended claims is not necessarily limited to the specific features or acts described.

What is claimed is:

1. A method, comprising:
receiving, at a first device and from a second device, an indication of a presence of the second device;
receiving, at the first device and from the second device, a first identifier unique to the second device;
sending, from the first device and to a server, a first request, including the first identifier, for a patient identifier uniquely identifying a patient associated with the first identifier;
receiving, at the first device and from the server, the patient identifier;
receiving, at the first device and from a third device, a second request for the patient identifier;
determining that the third device is within a threshold distance from the first device; and
based on the second request, and on determining that the third device is within the threshold distance, sending the patient identifier from the first device to the third device.

2. The method of claim 1, wherein sending the patient identifier to the third device further comprises:
scanning, with a scanning component of the first device, an identifier associated with the third device;
receiving, at the first device and based on scanning the identifier associated with the third device, a request for the patient identifier from the third device; and
sending, from the first device and based on receiving the request for the patient identifier, the patient identifier to the third device.

3. The method of claim 1, further comprising:
receiving, from a fourth device, a third request for the patient identifier;
based on the third request, determining that the third device is within the threshold distance from the first device;
based on the third request, determining that the fourth device is within the threshold distance from the first device; and
based on determining that the third device and fourth device are within the threshold distance, sending, from the first device or the third device, the patient identifier to the fourth device.

4. The method of claim 1 further comprising:
receiving, at the third device, biological data associated with the patient;
sending, from the third device to the server, the biological data; and
receiving, at the third device, based on sending the biological data, and from the server, an updated patient identifier comprising the biological data.

5. The method of claim 1, wherein determining that the third device is within the threshold distance of the first device comprises:
receiving, at the first device and from the third device, a first signal;
determining, by the first device and based on the first signal, a distance between the first device and the third device; and
determining, by the first device, that the distance between the first device and the third device is less than or equal to the threshold distance;
wherein the patient identifier is sent to the third device is based at least in part on determining that the distance between the first device and the third device is less than or equal to the threshold distance.

6. The method of claim 1, further comprising:
receiving, at the first device and from the server, a notification of an update to the patient identifier;
sending, from the first device and to the server, a request for the updated patient identifier;
receiving, at the first device, the updated patient identifier; and
causing presentation, via a user interface of the first device, of the updated patient identifier.

7. The method of claim 1, wherein the second device is an RFID tag, a barcode, a two-dimensional matrix barcode, a real-time location system tag, or a global positioning tag.

8. The method of claim 1, wherein the first device comprises a tag reader, and wherein receiving the first identifier from the second device comprises the first device receiving a signal from the second device.

9. The method of claim 1, wherein the patient identifier includes at least one of:
a name of the patient;
a date of birth of the patient;
a gender of the patient;
an age of the patient;
a medication list of the patient;
a room number of a patient;
a location of the patient;
a health history of the patient;
an admit status of the patient;
a medical record number; or
a visit number.

10. The method of claim 1, further comprising:
sending, from the first device and to the server, a third request for the patient identifier;
receiving, from the server, by the first device, and based on the third request, a disassociation indication; and
removing, based on the disassociation indication and from a memory of the first device, the first identifier and the patient identifier.

11. A system, comprising:
one or more processors; and
one or more computer-readable media storing instructions that, when executed by the one or more processors, cause the one or more processors to perform operations comprising:
receiving, at a first device and from a second device, an indication of a presence of the second device;
receiving, at the first device and from the second device, a first identifier unique to the second device;
sending, from the first device and to a server, a first request including the first identifier for a patient identifier uniquely identifying a patient and associated with the first identifier;
receiving, at the first device and from the server, the patient identifier;
receiving, at the first device and from a third device, a second request for the patient identifier;
determining, based on the second request, that the third device is within a threshold distance from the first device; and
based on determining that the third device is within the threshold distance, sending the patient identifier from the first device to the third device.

12. The system of claim 11, wherein sending the patient identifier to the third device further comprises:
scanning, with a scanning component of the first device, an identifier associated with the third device;
receiving, at the first device and based on scanning the identifier associated with the third device, a request for the patient identifier; and
sending, from the first device and based on receiving the request for the patient identifier, patient information to the third device.

13. The system of claim 11, the operations further comprising:
receiving, from a fourth device, a third request for the patient identifier;
based on the third request, determining that the third device is within the threshold distance from the first device;
based on the third request, determining that the fourth device is within the threshold distance from the third device; and
based on determining that the third device is within the threshold distance, sending, from the first device or the third device, the patient identifier to the fourth device.

14. The system of claim 11, wherein determining that the third device is within the threshold distance of the first device comprises:
receiving, at the first device and from the third device, a first signal;
determining, by the first device and based on the first signal, a distance between the first device and the third device; and
determining, by the first device, that the distance between the first device and the third device is less than or equal to the threshold distance;
wherein the patient identifier is sent to the third device is based at least in part on determining that the distance between the first device and the third device is equal to or less than the threshold distance.

15. A method, comprising:
scanning, with a first device, a tag of a second device comprising;
receiving, at the first device and from the second device, a first identifier unique to the tag of the second device;
sending, from the first device and to a server, a first request for a patient identifier uniquely identifying a patient associated with the first identifier;
receiving, at the first device and from the server, the patient identifier;
scanning, with a scanning component of the first device, a second identifier associated with a third device;
receiving, at the first device and based on scanning the second identifier associated with the third device, a second request for the patient identifier from the third device; and
sending, from the first device and based on receiving the second request for the patient identifier, the patient identifier to the third device.

16. The method of claim 15, wherein the tag is a radio frequency identification (RFID) tag, a barcode, a two-dimensional matrix barcode, Bluetooth tag, a real time location system (RTLS) tag, or a geolocation tag.

17. The method of claim 16, wherein the tag comprises a unique identifier associated with a patient in the server.

18. The method of claim 15, wherein the patient identifier includes at least one of:
a name of the patient;
a date of birth of the patient;
a gender of the patient;
an age of the patient;
a room number of a patient;
a medication list of the patient;
a location of the patient;
a health history of the patient;
an admit status of the patient;
a medical record number; or
a visit number.

19. The method of claim 15, further comprising:
recording, via the first device, biological data of the patient;
sending, from the first device to the server, the biological data; and
receiving, at the first device based on sending the biological data and from the server, an updated patient identifier comprising the biological data.

20. The method of claim 15, further comprising:
receiving, at the first device, a third request for the patient identifier from a fourth device;
determining, based on the third request from the fourth device, that the fourth device is within a threshold distance from the first device; and
based on determining that the fourth device is within the threshold distance from the first device, sending the patient identifier from the first device to the fourth device.

21. A method of associating and disassociating a patient identifier with multiple devices using a server, the method comprising determining that at least two devices of the multiple devices are within a threshold distance of one another; and sending the patient identifier to at least one of the multiple devices via the server.

22. The method of claim 21, wherein at least one of the at least two devices is:
a smart device;
a wheelchair;
a hospital bed;
a vital signs monitor;
an infusion pump;
a blood pressure monitor;
a blood glucose monitor;
a pulse oximeter; or
an electrocardiogram (ECG) monitor.

23. The method of claim 21, wherein determining that the at least two devices of the multiple devices are within the threshold distance is based at least in part on a radio-identification signal generated by one of the at least two devices.

24. A method of associating a patient identifier of a patient with multiple devices using a server, the method comprising associating a first device with the patient identifier via the server and sending the patient identifier from the first device to a second device based on determining that the second device is within a threshold distance of the first device.

25. The method of claim 24, wherein determining that the second device is within the threshold distance of the first device is based at least in part on determining, by the first device, that a distance based at least in part on a radio-identification signal from the second device is less than or equal to the threshold distance.

26. The method of claim 24, wherein the patient identifier comprises at least one of:
a name of the patient;
a date of birth of the patient;
a gender of the patient;
an age of the patient;
a room number of a patient;
a medication list of the patient;
a location of the patient;
a health history of the patient;
an admit status of the patient;
a medical record number; or
a visit number.

27. A method comprising:
receiving, by a first device, from a component worn by a patient, and based on the component being disposed less than a first threshold distance from the first device, information unique to the component;
providing, by the first device, the information to a server separate from the first device.

28. The method of claim 27 further comprising:
receiving, by the first device, from the server, and based on providing the information, a patient identifier unique to the patient; and
providing, by the first device, the patient identifier to a second device based on the second device being disposed less than a second threshold distance from the first device.

29. The method of claim 28, wherein at least one of the first device and the second device includes at least one of:
a smart device;
a wheelchair;
a hospital bed;
an infusion pump;
a vital signs monitor;
a blood pressure monitor;
a blood glucose monitor;
a pulse oximeter; or
an electrocardiogram (ECG) monitor.
